(19) United States
(12) Patent Application Publication
(10) Pub. No.: US 2024/0423947 A1
(43) Pub. Date: Dec. 26, 2024

(54) METHODS AND COMPOSITIONS FOR TREATING ANAL FISSURE WITH TOPICAL CREAM

(71) Applicant: Shahriar Sedghi, Macon, GA (US)

(72) Inventor: Shahriar Sedghi, Macon, GA (US)

(21) Appl. No.: 18/827,736

(22) Filed: Sep. 7, 2024

Related U.S. Application Data
(63) Continuation of application No. 18/439,237, filed on Feb. 12, 2024.
(60) Provisional application No. 63/444,895, filed on Feb. 10, 2023.

(51) Int. Cl. A61K 31/337; A61K 9/00; A61K 9/06; A61K 31/167; A61K 31/5513

(52) U.S. Cl. CPC A61K 31/337; A61K 9/0014; A61K 9/06; A61K 31/167; A61K 31/5513

(57) ABSTRACT

The present invention relates to methods and compositions for treating Anal Fissure by administering a therapeutic dose of the antiviral drug Paclitaxel. The proposed method has been found to be effective in reducing the symptoms of Anal Fissure, including inflammation, bleeding, tearing, scar tissue, as well as reducing the bacterial load associated with Anal Fissure. This invention provides a safe and effective alternative to current Anal Fissure treatment methods and compositions and has the potential to improve the health of millions of people suffering from Anal Fissure worldwide.

### CROSS REFERENCE TO RELATED APPLICATIONS

This application is a continuation of U.S. patent application Ser. No. 18/439,237, filed Feb. 12, 2024, which claims the benefit of U.S. provisional 63/444,895, filed on Feb. 10, 2023, the entirety of which is incorporated by reference.

### FIELD OF THE INVENTION

This invention relates to the field of health and medicine, specifically to the treatment of Anal Fissure.

### BACKGROUND

Anal fissures are small tears in the skin of the anus that cause severe pain and discomfort. Currently medication may be prescribed, such as topical nitrates, calcium channel blockers, or botulinum toxin and in some severe cases, surgery may be necessary to correct the underlying problem and relieve symptoms.

Anal fissures can also cause complications such as chronic pain, infection, and rectal bleeding and they can linger long term in a chronic state. Anal fissures aren't associated with more serious diseases, such as bowel cancer, although cancer of the anus may mimic an anal fissure. Some of the possible complications of an anal fissure include Chronic anal fissure where the tear fails to heal. Over time, this can cause extensive scar tissue at the site of the fissure (sentinel pile).

People with anal fissures may also experience difficulty passing stool, which can result in further discomfort and damage to the affected area. In order to prevent anal fissures and reduce their severity, it is important to maintain healthy bowel habits and to seek prompt treatment if symptoms develop.

### BRIEF DESCRIPTION OF THE INVENTION

The present invention introduces a novel method for treating Anal Fissure by administering a therapeutic dose of the drug Paclitaxel, also known by the brand names, taxol and Onxol. Paclitaxel is an established medication, primarily known for its use in coronary stents to prevent restenosis and in the management of strictures associated with fibrosis. The innovative application of paclitaxel in the treatment of Anal Fissures represents a significant departure from its traditional uses, leveraging its pharmacological properties to address a new range of clinical symptoms.

This method capitalizes on the unique antiviral, anti-fibrotic and anti-inflammatory properties of paclitaxel in a new use case, providing a dual-action approach to treatment of anal fissure. By addressing both the symptomatic relief of Anal Fissures and the underlying causes, such as bacterial infection and inflammation, this method offers a comprehensive therapeutic strategy. A proposed method according to an embodiment described herein alleviates the immediate discomfort associated with anal fissures and promotes their long-term healing, thereby reducing the incidence of recurrence and the need for surgical intervention, for example by providing an anti-spasmodic effect on the anal sphincter provided by co-administration of paclitaxel with diazepam cream which helps with the healing of the fissure by prevention of spasm.

It can be appreciated that a method of topical administration of Paclitaxel in combination with valium cream provides a convenient and effective treatment option for patients with anal fissure.

This invention provides a safe and effective alternative to current Anal Fissure treatment methods and compositions and has the potential to improve the health of millions of people suffering from Anal Fissure worldwide.

### DETAILED DESCRIPTION

In an embodiment of the present disclosure, a method is provided to treat a patient with Anal Fissure by topically administering a therapeutic dose of paclitaxel. The dose and frequency of administration will be determined based on the severity of the patient's Anal Fissure and may vary from patient to patient. The patient will be monitored for any adverse reactions and the efficacy of the treatment will be evaluated over time.

The proposed method offers a safe and effective alternative to existing Anal Fissure treatments and compositions, boasting multiple benefits. It notably reduces inflammation, bleeding, and promotes the healing of scar tissue, alongside diminishing the bacterial load associated with Anal Fissures. The side effects observed with this method are similar to those experienced with the current application of the drug in coronary stents, suggesting a favorable safety profile. This innovative approach has the potential to significantly improve the health and well-being of millions of individuals suffering from Anal Fissures around the globe, marking a substantial advancement in the management of this condition.

### Topical Cream

According to one embodiment of the present disclosure, paclitaxel is applied topically to a patient, either alone or combined with a cream containing valium (generic name: diazepam), for the treatment of anal fissures. This method of topical application involves the use of a gel or ointment formulation that contains the appropriate dose of paclitaxel, which is then directly applied to the affected area of the patient.

It can be appreciated that Valium (diazepam) serves as a muscle relaxant, aiding in the relaxation of the sphincter to facilitate healing of the fissure, whereas Paclitaxel functions as an anti-fibrotic agent. In one embodiment of the present disclosure, a patient's sphincter is initially dilated using a dilator before applying the topical cream. Alternatively, in another embodiment, the cream containing diazepam is applied first, followed by the application of the Paclitaxelcontaining cream.

This method of delivery is particularly useful for patients with moderate to severe anal fissure and allows for a localized release of the drug to the affected area, reducing the risk of systemic side effects. The addition of valium cream to the mixture enhances the antiinflammatory effect of Paclitaxel and provides additional pain relief for the patient.

The precise proportion of Paclitaxel to Valium (diazepam) cream will be tailored to the severity of the patient's anal fissure, and this ratio may differ from one patient to another. In one specific embodiment, the ratio of Paclitaxel to Valium in the topical formulation is established at a one-to-one ratio. In a different embodiment, this ratio is adjusted to two parts of Paclitaxel for every one part of Valium. Furthermore, according to another embodiment, the ratio of Paclitaxel to Valium within the topical mixture ranges from one-to-one to three-to-one, allowing for flexibility based on individual patient needs and the specific characteristics of the anal fissure being treated.

According to an embodiment of the present disclosure the dose of Paclitaxel for the treatment of Anal Fissure according to this invention is 0.5-30 mg per application. According to an embodiment of the present disclosure the dose of Paclitaxel for the treatment of Anal Fissure according to this invention is 5-20 mg per application. This dosage is integrated into the topical cream and is to be applied on a weekly basis, daily, or following the patient's bowel movement, depending on the specific treatment protocol. For instance, the treatment protocol may vary the dosage frequency from a daily application of 0.5 mg to a more concentrated 30 mg dosage applied monthly. This flexibility allows for customization of the treatment plan to best suit the patient's needs and the severity of the Anal Fissure.

The frequency of application may be adjusted according to the severity of the case. According to an alternative embodiment it is administered every week for a less severe case. According to an alternative embodiment the dose of Paclitaxel is between 20-100 mg as part of the topical cream, administered every week, daily, or after a patient's bowel movement. The topical cream containing Paclitaxel, or Paclitaxel together with valium, can be manufactured in a variety of doses to cover every possible severity of anal fissure and schedule of application. According to an embodiment, the cream is available in three different dose ranges: 5-20 mg, 20-100 mg, 100-200 mg, and 200-500 mg of Paclitaxel per application. For mild to moderate anal fissure, a low-dose topical cream in the range of 5-20 mg may be provided according to an embodiment, with a recommended application of 1-2 times per week, or after a bowel movement of patient. For more severe cases of anal fissure, a higher dose topical cream in the range of 100-200 mg or 200-500 mg of Paclitaxel may be provided according to an embodiment, with a recommended application 7 per week.

The exact dose of the topical cream can be determined based on the severity of the patient's anal fissure and the patient's response to treatment, according to an embodiment of the present disclosure.

The frequency of application will also be adjusted based on the patient's response to treatment, with the goal of maintaining a therapeutic level of Paclitaxel in the affected area to effectively treat anal fissure.

According to an embodiment topical cream can be easily applied using an applicator or directly, providing a convenient and effective treatment option for patients with anal fissure. According to an embodiment it is applied after a dilation. The cream may be applied by an applicator or by a finger of the patient.

The dose and frequency of administration will be determined based on the severity of the patient's Anal Fissure and may vary from patient to patient. However, the dose administered every two weeks is the starting point for most patients and can be adjusted as needed based on the patient's response to treatment and any adverse reactions that may occur. The topical cream allows for a slow and steady release of the drug into the patient's affected anal fissure, providing a consistent level of Paclitaxel to the patient's affected area to effectively treat the Anal Fissure.

According to an alternative embodiment, Paclitaxel is applied to a patient topically, either alone or in a mixture with valium cream, to treat the patient's anal fissure cause by Crohn's disease.

Crohn's disease is a type of inflammatory bowel disease that can cause chronic inflammation in the gastrointestinal tract. The inflammation caused by Crohn's disease can lead to the development of anal fissures. The inflammation caused by Crohn's disease can weaken the tissues in the anus, making them more prone to fissure development. The preparation described herein can be used to treat a patient with anal fissure caused by Crohn's disease.

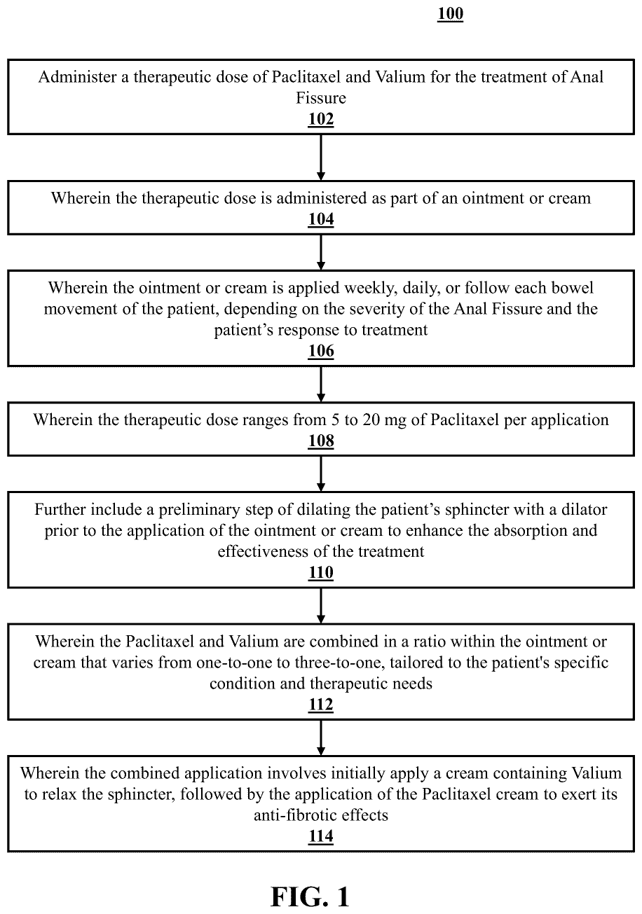

FIG. 1 is a flowchart of an example process 100 of treating anal fissure. In some implementations, one or more process blocks of FIG. 1 may be performed by a device.

As shown in FIG. 1, process 100 may include administering a therapeutic dose of Paclitaxel and Valium for the treatment of Anal Fissure (block 102).

Process 100 may include additional implementations, such as any single implementation or any combination of implementations described below and/or in connection with one or more other processes described elsewhere herein. In a first implementation, the therapeutic dose is administered as part of an ointment or cream (block 104).

In a second implementation, alone or in combination with the first implementation, the ointment or cream is applied weekly, daily, or following each bowel movement of the patient, depending on the severity of the Anal Fissure and the patient's response to treatment (block 106).

In a third implementation, alone or in combination with the first and second implementation, the therapeutic dose ranges from 5 to 20 mg of Paclitaxel per application (block 108).

A fourth implementation, alone or in combination with one or more of the first through third implementations, process 100 further including a preliminary step of dilating the patient's sphincter with a dilator prior to the application of the ointment or cream to enhance the absorption and effectiveness of the treatment (block 110).

In a fifth implementation, alone or in combination with one or more of the first through fourth implementations, the Paclitaxel and Valium are combined in a ratio within the ointment or cream that varies from one-to-one to three-to-one, tailored to the patient's specific condition and therapeutic needs (block 112).

In a sixth implementation, alone or in combination with one or more of the first through fifth implementations, the combined application involves initially applying a cream containing Valium to relax the sphincter, followed by the application of the Paclitaxel cream to exert its anti-fibrotic effects (block 114).

Although FIG. 1 shows example blocks of process 100, in some implementations, process 100 may include additional blocks, fewer blocks, different blocks, or differently arranged blocks than those depicted in FIG. 1. Additionally, or alternatively, two or more of the blocks of process 100 may be performed in parallel.

In an innovative extension of the present treatment methodology for Anal Fissure, the incorporation of Lidocaine, known generically as xylocaine, into the therapeutic formulation offers a multifaceted approach to patient care. Lidocaine, a well-established local anesthetic, is introduced as a third active ingredient alongside Paclitaxel and Valium (diazepam), within the ointment or cream. This strategic addition is designed to provide immediate symptomatic relief from pain, a common and distressing symptom of Anal Fissures. The presence of Lidocaine not only enhances patient comfort during the healing process but also complements the musclerelaxant properties of Valium (diazepam) and the anti-fibrotic effects of Paclitaxel. By addressing the immediate pain, facilitating muscle relaxation, and promoting tissue repair, this tri-component formulation represents a comprehensive and patient-centric approach to the treatment of Anal Fissure. The synergistic effects of Paclitaxel, Valium (diazepam), and Lidocaine (xylocaine) within a single topical application underscore the commitment to optimizing therapeutic outcomes and improving the quality of life for patients suffering from this condition.

1. A method of treating Anal Fissure in a patient, comprising: administering a therapeutic dose of Paclitaxel and Valium for the treatment of Anal Fissure, wherein the therapeutic dose is administered as part of an ointment or cream.

2. The method of claim 1, wherein the therapeutic dose ranges from 5 to 20 mg of Paclitaxel per application.

3. The method of claim 2, wherein the ointment or cream is applied following each bowel movement of the patient.

4. The method of claim 1, further including a preliminary step of dilating the patient's sphincter with a dilator prior to the application of the ointment or cream.

5. The method of claim 1, wherein the Paclitaxel and Valium are combined in a ratio within the ointment or cream that varies from one-to-one to three-to-one.

6. The method of claim 6, wherein the combined application involves initially applying a cream containing Valium to relax the sphincter followed by the application of the Paclitaxel cream.

7. The method of claim 7, further comprising administering Lidocaine as an additional drug in the ointment or cream.

8. A topical formulation specifically designed for the treatment of Anal Fissure, comprising
a combination of a therapeutic dose of Paclitaxel and Valium.

9. The formulation of claim 8, wherein the therapeutic of paclitaxel dose ranges from 5 to 20 mg of Paclitaxel per application.
